(19) United States
(12) Patent Application Publication
(10) Pub. No.: US 2013/0197314 A1
(43) Pub. Date: Aug. 1, 2013

(54) Speculum

(71) Applicant: Paul Eakin, County Down (IE)

(72) Inventor: Paul Eakin, County Down (IE)

(73) Assignee: PELICAN HEALTHCARE LIMITED, Llanishen, Cardiff (UK)

(21) Appl. No.: 13/810,910

(22) PCT Filed: Feb. 18, 2011
(86) PCT No.: PCT/GB2011/050323

§ 371 Date: Mar. 29, 2013

(30) Foreign Application Priority Data
Jul. 21, 2010 (GB) 1012224.0

(51) Int. Cl. A61B 1/32

(52) U.S. Cl. CPC A61B 1/32
USPC 600/203; 156/247

(57) ABSTRACT

A speculum is provided, comprising two or more blades moveable between an open and a closed position. A tubular sheath is attached to at least one of the blades, the sheath being arranged to retain the lateral walls of the body cavity when the blades are in the open position. Sheath attachment means provided on one of the speculum blades facilitate attachment of the sheath to the blade, the attachment means comprising at least one patch or strip of adhesive material extending in a longitudinal direction of the blade.

### CROSS-REFERENCE TO RELATED APPLICATIONS

This application claims priority from PCT/GB/2011/050323 filed on Feb. 18, 2011 and from GB 1012224.0, filed Jul. 21, 2010, which are hereby incorporated by reference in their entireties.

### BACKGROUND OF THE INVENTION

1. Field of the Invention

This invention relates to the field of medical tools, in particular to specula.

2. State of the Art

A speculum is a commonly-used medical tool for opening or distending an orifice or cavity of a patient to permit examination of the interior and/or to enable a sample to be taken.

Various forms of specula are known, their designs varying in accordance with the body cavity to be inspected (e.g. vagina, rectum, ear, nostril). However, all forms of specula incorporate the same basic concept: elongate separator elements (‘paddles’, or ‘blades’), typically having a curved cross section, are positioned edge to edge so that in combination they form an elongate channel. The channel narrows towards the end to be inserted into the orifice (the distal end) to aid the insertion process whilst remaining relatively wider at the opposing (proximal) end to facilitate visual examination or insertion of an instrument via the mouth of the speculum.

The two blades are hinged at their broader ends, thus resembling a duck's beak. Thus, the speculum is able to move to move between a closed configuration (i.e. blade edges are adjacent to each other) and an open configuration (i.e. blades are angled away from each other). Handles are attached, usually integrally, at the hinged ends of the blades. Squeezing of the handles towards each other causes the blades to move apart thus dilating the orifice or cavity and enabling examination. When the examination is complete, the handles are moved apart from each other, thus allowing the blades to move towards each other and into the closed configuration such that the speculum can be withdrawn from the cavity.

It is not uncommon for excess tissue to be present in the cavity. In the case of the vagina, for example, women who have had several pregnancies, a prolapsed uterus or other uterine or vaginal damage may have excess vaginal tissue. Obese women may also have an increase in the loose connective tissue beneath the pelvic peritoneum.

In such cases, when such patients have a vaginal speculum examination, the loose vaginal walls may collapse inwards (i.e. towards the midline of the speculum blades) as the blades attempt to keep the anterior and posterior walls apart. The collapse of the vaginal walls results in an obscuring of the visualisation of the cervix for the purpose of cervical cultures, ruptured membranes, pap smears, biopsy or the visual assessment of the degree of dilation.

The problem of vaginal wall collapse has been addressed in known specula. For example, U.S. Pat. No. 6,432,048 discloses a speculum having including a flexible sheath which is configured to expand during opening of the blades but collapse into a folded configuration when the blades are closed, the folded sheath being held between the closed blades. Thus, when the speculum is pushed into the cavity, the sheath may be pushed towards the mouth of the speculum, and so pockets are provided around the distal tips of the blades to prevent such movement. Moreover, upon withdrawal of the speculum, the sheath may slide off the blades unless the blades are closed sufficiently tightly as to clamp the folded sheath therebetween.

GB 2424585 discloses a speculum having a flexible wall member attached to the distal end of at least one of the blades by means of adhesive. When the blades are opened the wall member expands. It then retains this expanded shape when the blades are subsequently closed. Thus, the wall member does not return to its original formation and remains in an expanded or stretched state. Upon withdrawal of the speculum, the wall member is refracted back along the blades by friction contact with the body cavity.

U.S. Pat. No. 6,902,530 discloses an elastic mesh covering for a speculum, the cover being attached to the speculum by means of strings which the practitioner must tie around the back of the device before use and untie after use. The strings may be fiddly to use, may obscure the practitioner's view during use, and may hinder the use of instruments at the mouth of the speculum. Moreover, the cover extends over the length of both blades and partially obscures the practitioner's view via the open distal ends.

U.S. Pat. No. 5,787,648 also shows a sheath for use with a speculum. However, the sheath is not attached to the blades and also retains its shape when the blades are closed and partially withdrawn. Similarly, U.S. Pat. No. 6,036,638 shows a speculum sleeve which is not attached to the speculum.

U.S. 2005/0192482 A1, U.S. 2008/0242938 A1, U.S. Pat. No. 6,432,048 B1, U.S. Pat. No. 3,841,317 and U.S. 2003/0069477 A1 all disclose known arrangements for attaching a sheath or membrane to a speculum blade by means of adhesive.

### SUMMARY OF THE INVENTION

An improved arrangement has now been devised.

Thus, according to a first aspect of the invention, there is provided a speculum comprising
two or more blades moveable between an open and a closed position;
a sheath attached to at least one of the blades, the sheath being arranged to retain the lateral walls of the body cavity when the blades are in the open position; and
sheath attachment means for attaching the sheath to a blade of the speculum, the attachment means comprising a patch or elongate strip of adhesive material.

Thus, in a preferred embodiment, the speculum carries a flexible sheath or cover which is preferably tubular. The sheath is preferably a liquid impermeable membrane and may be constructed from a plastics material, such as polyurethane. Preferably, the sheath is relatively thin. Preferably, the membrane thickness of the membrane is in the range of 20 to 50 mm.

The sheath may be substantially circular in cross section. Alternatively or additionally, the sheath may taper in diameter towards one end such that it is narrower at that end. However, the sheath may, in some embodiments, have a constant diameter along its longitudinal length.

Preferably, the sheath is open at both ends to enable insertion of the speculum blades through both open ends of the sheath.

In a preferred embodiment, the sheath is open-ended and covers the speculum by encircling an area of the blades between their distal and proximal ends. Preferably, the sheath encircles a mid-section of the blades such that it extends along only a portion of the blades lengths. Thus, the sheath may not extend along the entire length of the blade. Alternatively, the sheath may cover substantially the entire length of the blades.

The sheath may be removably or permanently attached to the speculum.

The attachment means is a patch or area of adhesive material. The patch of adhesive material is an elongate area of adhesive, such as a strip, and may be glue. Alternatively, the area of adhesive may be a line or strip of glue, or a series of dots or blobs. Preferably, the adhesive material is provided towards or on the mid-line of the speculum blade, such as the apex of a curved speculum blade. Preferably, the strip extends in the longitudinal direction of the blade. This provides the advantage of a secure attachment of the sheath to the blade surface.

Preferably, the adhesive material is provided on the outer surface of a blade to facilitate attachment of the sheath. Thus, in use, the adhesive material sits between the sheath and the blade, holding the sheath in secure relation to the speculum. The adhesive strip provides the advantage that whilst providing a secure attachment of the sheath to the speculum, it is also relatively flat or shallow in profile and so does not stand proud of the blade to any great degree. Thus avoids causing damage to the cavity tissue or discomfort to the patient.

Preferably, the adhesive strip, path or line is spaced from the terminal edges of the sheath such that the adhesive is not present at the terminal edges. Thus, the adhesive strip may extend along the entire longitudinal length of the sheath or, more preferably, extend only partially along the length of the sheath. Similarly, the adhesive strip may not extend width-wise to the longitudinal edges of the speculum blades. Thus, the area of sheath/blade around the adhesive strip may not be exposed to the adhesive material. This provides the advantage that only a relatively small amount of adhesive is required to achieve a secure attachment to the blade, thus lowering manufacturing costs.

Preferably, during manufacture, the adhesive strip is provided with a removable protective member. This protective member may be a peelable cover or shield which is provided over the adhesive prior to application of the sheath in order to protect the adhesive from damage and/or prevent accidental adhesion of to the sheath whilst the sheath is being positioned on the speculum. The protective member may be folded over upon itself substantially in a U-shape. One arm of the U may be longer than the other to facilitate gripping of the arm such that a user can grasp the protective member and peel it away from the adhesive. The sheath can be positioned on the speculum with the protective member in place, and the protective member can then be peeled back in order to allow the adhesive present on the speculum to contact and secure the sheath. This provides the advantage that the sheath can be applied to the speculum with ease, thus facilitating assembly of the sheath and speculum.

The adhesive may be such that it can withstand multiple uses i.e. the sheath may be applied to the blade more than once, and/or the peel-off strip may be re-applied more than once, without the attachment means losing its adhesive qualities.

In some embodiments of the invention, the speculum may be provided with the sheath already attached to the speculum prior to use. It is generally preferred, however, that the adhesive strip and sheath are provided separately from the speculum for assembly prior to use on a patient.

In one embodiment, the attachment means may be a double-sided adhesive strip wherein the first side of the adhesive strip is configured for adhesion to the speculum blade and the second side is configured for adhesion to the sheath.

Preferably, a plurality of attachment means is provided. Thus, an adhesive strip may be provided on each blade. This provides an even more secure attachment and reduces or eliminates the possibility of the sheath becoming unattached from the speculum during use. It also provides the advantage that the sheath is less likely to tear, slip, wrinkle or bunch up during insertion or withdrawal of the speculum from the body because both the top and bottom of the sheath are held in position on the blades.

Preferably, the sheath is substantially transparent. Thus, the practitioner's view of the cavity walls is not impeded by the sheath when using it with a transparent (e.g. plastic) speculum.

Preferably, the sheath is a solid tube or band of elastic material, or, in some embodiments, may have recesses or apertures formed in the body of the sheath. The sheath may be a mesh.

Preferably, the sheath is biased to an unexpanded, initial configuration (e.g. shape and size) which it maintains when the speculum blades are closed, but is stretched into an expanded configuration when the speculum blades move away from one another into an open position. The speculum blades may push against the resilience of the sheath as the blades open. As the blades return from an open to a closed position, the sheath contracts such that it returns to and retains its original, unexpanded form. Thus, the sheath does not collapse, balloon or sag after the blades have been opened.

This provides the advantage that the contracted sheath does not interfere with or collapse between the blades when they are closed. Furthermore, if the blades are re-opened the sheath may maintain substantially its original resilience and continue to urge the blades back towards each other as it did upon first use.

According to the present invention there is also provided a method of assembling a speculum, wherein the speculum has two or more blades moveable between an open and a closed position, the method comprising the steps:
i) positioning an adhesive member along the longitudinal axis of a blade;
ii) positioning a sheath around the speculum blades such that the sheath is held in position by the adhesive member, the sheath being arranged to retain the lateral walls of the body cavity when the blades are in the open position.

Preferably, the adhesive member is a double-sided, elongate adhesive strip.

Preferably, one or both sides of the adhesive member are protected by a peel-off shield.

These and other aspects of the present invention will now be described by way of example only and with reference to the accompanying drawings.

### BRIEF DESCRIPTION OF THE DRAWINGS

FIG. 1 is a side view of a typical speculum, the speculum being in a closed configuration;

FIG. 2 shows a side view of a speculum in accordance with a preferred embodiment of the invention, the speculum being in the closed position and the sheath attached to both the top and the bottom blades.

FIG. 3 shows the speculum of FIG. 2 in the open position.

FIG. 4 shows an alternative embodiment of the invention in which the sheath is attached only to the top blade of the speculum.

FIG. 5 shows an alternative embodiment of the invention in which the sheath is attached only to the bottom blade of the speculum.

FIG. 6 shows a detailed view of the adhesive strip, protective shield and sheath provided on the speculum in accordance with an embodiment of the invention.

FIG. 7 shows a plan view of a speculum in accordance with the present invention.

### DETAILED DESCRIPTION OF THE PREFERRED EMBODIMENTS

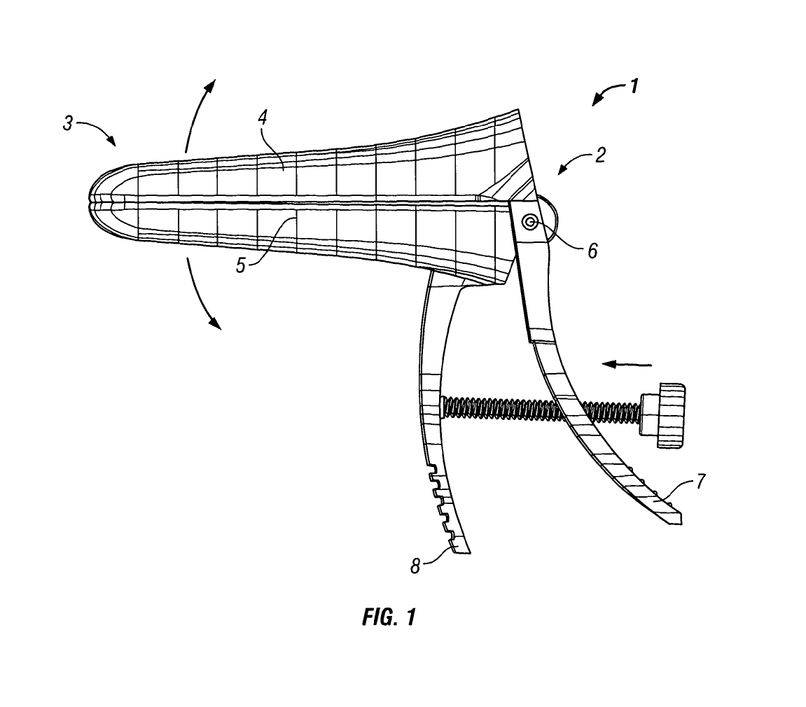

Referring initially to FIG. 1, there is shown a vaginal speculum (1) of the type commonly used for dilating the vaginal cavity or orifice of a patient during examination or medical procedure. Such specula are typically manufactured from a plastic or metal material, having a plurality of curved separator elements typically referred to as ‘blades’ or ‘paddles’. In a typical arrangement, there are two blades (4, 5) oriented such that one provides an upper blade (4) and the other provides a lower blade (5).

The end of the speculum which will be closest to the practitioner during use of the speculum is typically open and known hereafter as the ‘proximal end’ or ‘mouth’ (2). The body cavity may be inspected through the open proximal end or mouth (2). The other end of the speculum, which is inserted into the cavity, is known hereafter as the ‘distal end’ (3).

The blades (4, 5) are coupled such that they can move towards or away from each other. Depending on the coupling arrangement used, this may be angular or lateral movement. In a typical arrangement, as shown in FIG. 1, the coupling is a hinge (6) provided at the proximal end (2) of the speculum (1), such that the blades (4, 5) may pivot around the hinge (6) at their proximal ends (2) thus moving the distal ends (3) away from or towards each other. The open mouth (2) formed at the proximal end of the blades enables a practitioner to insert an examination or surgical device into the speculum through the mouth, and also enabling visual inspection of the cavity or body part.

Thus, the speculum 1 is moveable between a closed configuration (as shown in FIG. 1) or in an open (expanded) configuration in which there is angular separation displacement of the blades (4, 5).

The blades (4, 5) are provided, coupled integrally or otherwise, with handles (7, 8). Upper blade (4) is shown as coupled to rear handle (7), and lower blade (8) is coupled to front handle (8). Pivoting the handles (7, 8) together thus causes the blades (4, 5) to move apart into an open (expanded) configuration.

It should be noted that a number of different mechanisms exist for supporting the blades and the present invention is not limited in this regard. The present invention may be used in conjunction with a variety of types or styles of speculum.

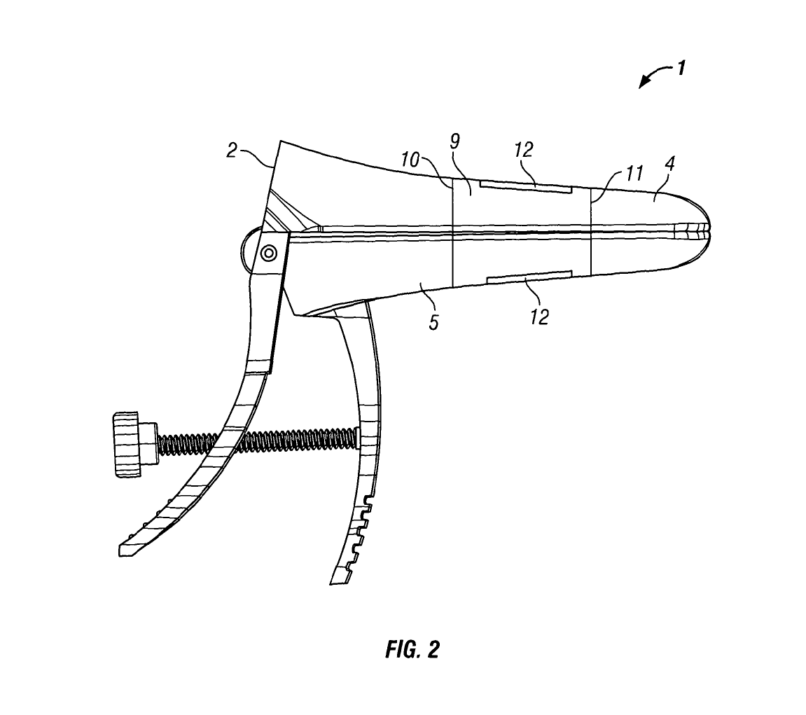

Turning to FIGS. 2 to 7, a speculum is provided according to the invention and has a polyurethane sheath (9) wrapped around a portion of the blades (4, 5). The polyurethane sheath (9) is generally tubular in shape. A sheath of thickness 30 mm has been shown to provide ideal operational characteristics.

The sheath (9) has two ends: a proximal (10) end which, in use, is oriented towards the proximal end of the blades 2; and a distal end (11) which, in use, is oriented towards the distal end of the blades (3). Both ends (10, 11) of the tubular sheath (9) are open to enable the speculum blades to pass through both ends. The distal end 11 of the sheath (9) has a slightly smaller diameter than that of the proximal end (10), such that the sheath (9) is tapered. The nature of the elastic sheath (9) is such that the sheath conforms to and clings to the speculum (1) in a skin-like fashion.

When the speculum 1 is closed as shown in FIG. 2, the sheath (9) is in its initial, unexpanded form. The sheath may be arranged and configured to resiliently bias the blades (4, 5) towards the closed position, although the urging force and resilience of the sheath may vary according to a variety of factors, such as dimensions, material and so on. The sheath (9) is transparent or semi-transparent such that visualisation of the cavity is not impeded during use.

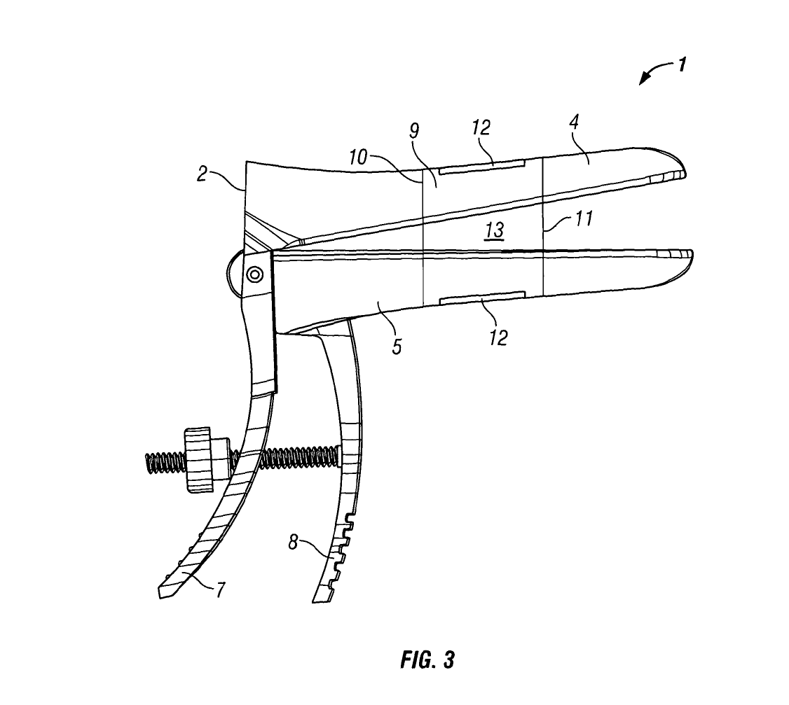

FIGS. 3, 4 and 5 show the speculum (1) in an open configuration. With the speculum in this position, the sheath (9) is stretched into an expanded form by the blades. Thus, the diameter of the sheath has increased at the distal end relative to its diameter when in its initial, contracted form. In this position, the resilient sheath may apply a force to the blades to urge them towards the closed position of FIG. 2.

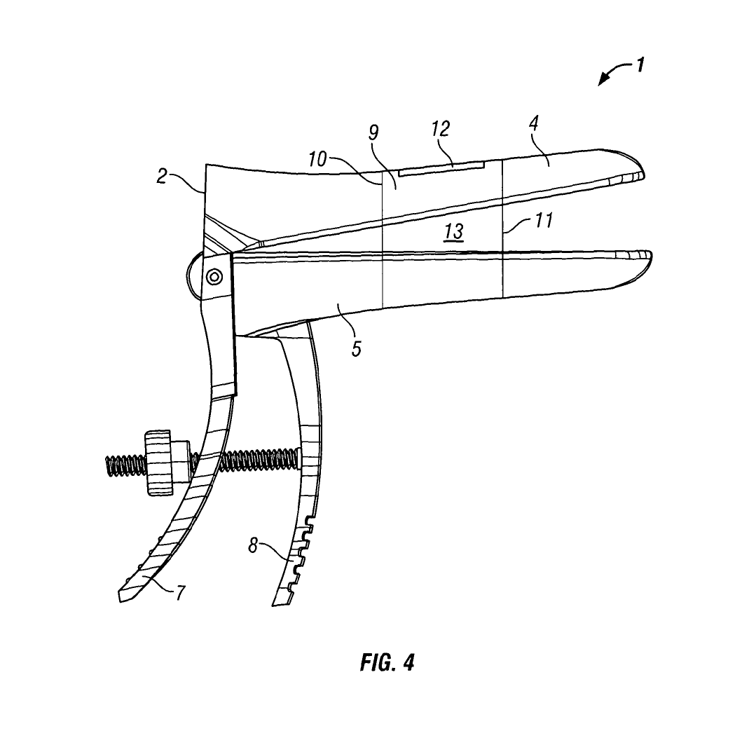

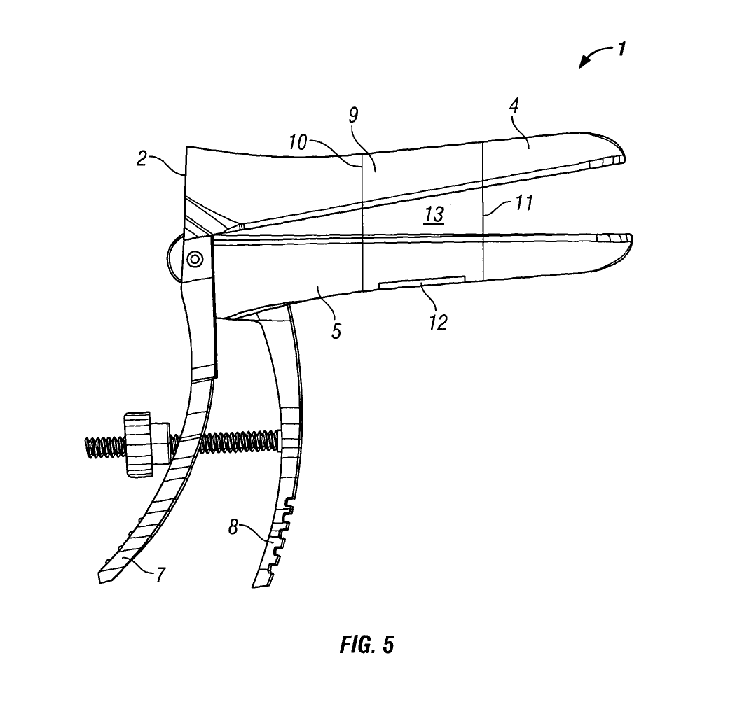

According to one embodiment of the invention, the sheath (9) is attached to or carried on at least one blade. FIGS. 2 and 3 show the sheath attached to the external surfaces of both the top and bottom blades (4, 5). However, alternative embodiments are shown in FIGS. 4 and 5 which show the sheath (9) attached to only the top (4) and bottom (5) blades respectively. The embodiment of FIGS. 2 and 3 is generally preferred because the attachment to both blades provides a more secure attachment of the sheath and reduces the likelihood that the sheath will rip, wrinkle or bunch up or detach from the speculum (1) during insertion/withdrawal into/from the body cavity.

The sheath (9) is attached to the blades by an adhesive strip (12) as shown in the Figures although other types of attachment means may be used to similar effect. The strip (12) is an elongate area of adhesive. However, other forms or shapes may be used. For example, a blob or dot of adhesive (or series of spaced blobs or dots of adhesive) may be used instead of an elongate, generally rectangular strip. However, the elongate strip (12) provides enhanced hold of the sheath (9) because a greater area of the sheath is attached to the blade.

The strip (12) extends in the longitudinal direction of the blade and/or sheath. If being used with a speculum having curved blades, the adhesive strip may be provided at the apex of the curve.

As best seen in FIG. 7, the adhesive strip is spaced from the terminal edges of the sheath 10, 11 such that the adhesive is not present at the edges (i.e. it does not extend along the entire longitudinal length of the sheath). Moreover, the adhesive strip does not extend width-wise to the longitudinal edges of the speculum blades. Thus, the area of sheath/speculum around the adhesive strip is not exposed to the adhesive material, there being a margin or non-adhered area formed around the strip which is not in contact with the adhesive.

The sheath may be removably attached to the speculum. In such embodiments, the adhesive is selected such that the flexible sheath can be detached and possibly re-attached (for example, for re-positioning of the sheath). In other embodiments, the sheath may only be attached to the speculum once, such that removal and/or re-attachment are not possible.

During assembly or manufacture, the adhesive strip (12) is attached to an external surface of the speculum blade (4). This may be performed during manufacture of the speculum, such that the speculum is already provided with the adhesive strip in position on the speculum blade. Alternatively, a double-sided adhesive strip may be provided separately from the speculum such that the practitioner applies one side of the adhesive strip to a speculum blade prior to fitting the sheath around the speculum on top of the adhesive strip.

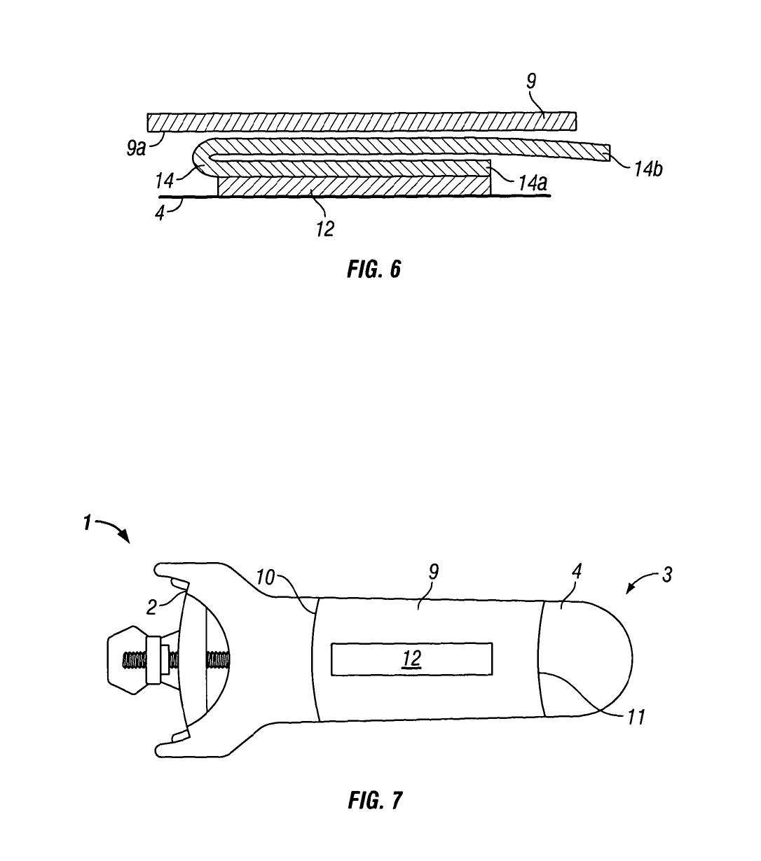

The adhesive strip (12) is protected by a substantially U-shaped, peel-off shield (14) which is folded over onto itself, having two arms (14a, 14b). This is shown in FIG. 6. The first arm (14a) of the U is applied to the adhesive strip to cover it. Thus, the adhesive strip is not exposed and cannot stick to the inner surface (9a) of sheath (9) during application of the sheath. The second arm (14b) rests on top of the first arm (14a). The second arm (14b) is of sufficient length to extend beyond the edge of the sheath when the sheath is applied, to enable the second arm (14b) to be easily accessible.

The polyurethane sheath (9) is then fitted around the speculum in the desired position. The second arm (14b) of the shield may then be gripped and pulled to peel the shield away from the adhesive strip (12), exposing it. The inner surface (9a) of the sheath is then brought into contact with the adhesive to attach the sheath securely to the speculum blade.

In cases where a double sided adhesive strip is used, a protective shield can be provided for each adhesive surface of the strip. The practitioner peels off the protective shield (14) from one side of the adhesive strip, and attaches the strip to the blade prior to peeling off the protective shield from the second side of the strip. The exposed second side of the adhesive strip then adheres to the internal surface of the sheath when the sheath (9) is fitted around the speculum (1). Thus, the adhesive strip is sandwiched between the sheath and the speculum blade.

During use (i.e. after manufacture and/or assembly as described above) the distal end (3) of the closed, sheathed speculum shown in FIG. 2 is inserted into the body cavity. The blades (4, 5) are then urged apart to open the speculum as per FIGS. 3, 4 and 5. As the blades open, they push against and retain the cavity walls to enable examination. During this process, the sheath (9) is stretched by the opening blades, thus providing two retaining walls or barriers (13) on either side of the speculum between the edges of the blades. The stretched sheath (9) prevents the cavity walls from collapsing into the speculum between the open blades so that the examination or medical procedure may be carried out without impediment, as most clearly seen in FIGS. 3, 4 and 5.

After the examination is complete, the practitioner allows the blades (4, 5) to return to their initial, closed position (as per FIG. 2) prior to withdrawing the speculum (1). As the blades close, the elastic sheath (9) contracts back to its original form and size. Thus, the sheath (9) is biased towards its initial, un-stretched configuration. Thus, sagging or ballooning of the sheath (9) is eliminated or at least minimised.

After use, the sheath (9) may be disposed of along with a disposable speculum. Alternatively, if the sheath is used with a non-disposable speculum, it may be removed and discarded. The non-disposable speculum may then be sterilised prior to re-use.

The present invention provides numerous advantages over the prior art solutions. These include the following. The elastic sheath:
does not impede or obscure the practitioner's view through the speculum or access to either end of the of the speculum;
does not impede or hinder use of accessories which may be mounted at the mouth or inside the speculum;
is cheap and easy to manufacture;
is quick and easy to use;
retains the cavity walls around the entire perimeter of the cavity;
helps prevent pinching of tissue between the closing blades;
may be retro-fitted to an existing speculum without adaptation of the speculum;
may be used with any type of speculum.
is easily fitted around and secured to the speculum by the practitioner prior to use
the size, orientation and/or position of the adhesive strip means that only a relatively small amount of adhesive is required, thus keeping manufacturing costs to a minimum, whilst providing a secure attachment of the sheath to the blade.

It should be noted that the above-mentioned embodiments illustrate rather than limit the invention, and that those skilled in the art will be capable of designing many alternative embodiments without departing from the scope of the invention as defined by the appended claims. In the claims, any reference signs placed in parentheses shall not be construed as limiting the claims. The word “comprising” and “comprises”, and the like, does not exclude the presence of elements or steps other than those listed in any claim or the specification as a whole. In the present specification, “comprises” means “includes or consists of” and “comprising” means “including or consisting of”. The singular reference of an element does not exclude the plural reference of such elements and vice-versa. In a device claim enumerating several means, several of these means may be embodied by one and the same item of hardware. The mere fact that certain measures are recited in mutually different dependent claims does not indicate that a combination of these measures cannot be used to advantage.

1. A speculum comprising:
two or more blades moveable between an open and a closed position;
a tubular sheath arranged to retain the lateral walls of the body cavity when the blades are in the open position; and
sheath attachment means provided on at least one of the blades for attaching the sheath to the at least one blade, the sheath attachment means comprising at least one patch or strip of adhesive material extending in a longitudinal direction of the at least one blade.

2. A speculum according to claim 1, wherein:
the sheath attachment means is provided with a removable protective member.

3. A speculum according to claim 2, wherein:
the protective member is substantially U-shaped.

4. A speculum according to claim 1, wherein:
the sheath is substantially transparent.

5. A speculum according to claim 1, wherein:
the sheath attachment means is spaced from the edges and/or ends of the at least one blade such that a non-adhered margin is formed around the sheath attachment means.

6. A speculum according to claim 1, wherein:
the sheath attachment means is spaced from the opposing ends of the sheath.

7. A speculum according to claim 1, wherein:
the sheath attachment means is a double-sided adhesive strip, the first side of the adhesive strip being configured for adhesion to the speculum blade and the second side being configured for adhesion to the sheath.

8. A speculum according to claim 7, wherein:
each side of the double-sided adhesive strip is provided with a protective shield.

9. A speculum according to claim 1, wherein:
the sheath attachment means is an elongate patch or strip of adhesive material, or a plurality of spaced blobs of adhesive material.

10. A speculum comprising:
two or more blades moveable between an open and a closed position; and
sheath attachment means provided on at least one of the blades blade for attaching a tubular sheath to the at least one blade, the sheath being arranged to retain the lateral walls of the body cavity when the blades are in the open position, wherein the sheath attachment means extends in the longitudinal direction of the at least one blade and comprises at least one area of adhesive material.

11. A speculum according to claim 10, wherein:
the sheath attachment means is provided with a removable protective member.

12. A speculum according to claim 11, wherein:
the protective member is substantially U-shaped.

13. A speculum according to claim 10, wherein:
the sheath attachment means is spaced from the edges and/or ends of the at least one blade.

14. A method of assembling a medical apparatus, comprising:
i) providing a speculum having two or more blades moveable between an open and a closed position, and an adhesive member provided along the longitudinal axis of at least one blade, the adhesive member being provided with a protective member;
ii) positioning a tubular sheath around the blades, the sheath being arranged to retain the lateral walls of the body cavity when the blades are in the open position; and
iii) removing the protective member from the adhesive member.

15. A method according to claim 14, wherein:
the protective member is peelable.

16. A method according to claim 14, wherein:
the protective member is substantially U-shaped.

17. A speculum according to claim 1, wherein:
the sheath covers a mid-section of the blades relative to the opposing ends of the blades.
